(19) United States
(12) Patent Application Publication
(10) Pub. No.: US 2021/0201698 A1
(43) Pub. Date: Jul. 1, 2021

(54) APPARATUS AND METHOD FOR SENSORY SUBSTITUTION

(71) Applicant: Electronics and Telecommunications Research Institute, Daejeon (KR)

(72) Inventors: Jun Seok Park, Daejeon (KR); Moo Seop Kim, Sejong-si (KR); Kyeong Deok Moon, Daejeon (KR); Yun Kyung Park, Daejeon (KR); Sung Yong Shin, Daejeon (KR); Hyung Cheol Shin, Daejeon (KR); Chang Mok Oh, Daejeon (KR); Chi Yoon Jeong, Daejeon (KR)

(73) Assignee: Electronics and Telecommunications Research Institute, Daejeon (KR)

(21) Appl. No.: 17/132,610

(22) Filed: Dec. 23, 2020

(30) Foreign Application Priority Data
Dec. 26, 2019 (KR) 10-2019-0175700

(51) Int. Cl. G09B 21/00; G06T 7/90; H04N 9/67

(52) U.S. Cl. CPC G09B 21/003; G06T 2207/10024; H04N 9/67; G06T 7/90

(57) ABSTRACT

A sensory substitution method includes obtaining a color image by using a sensor device, converting color information about the color image, received from the sensor device, into tactile information by using a user terminal, and generating a tactile stimulus corresponding to the tactile information received from the user terminal by using a tactile output device.

### CROSS-REFERENCE TO RELATED APPLICATIONS

This application claims priority under 35 U.S.C. § 119 to Korean Patent Application No. 10-2019-0175700, filed on Dec. 26, 2019, the disclosure of which is incorporated herein by reference in its entirety.

### TECHNICAL FIELD

The present invention relates to sensory substitution technology for substituting sensory information into other sensory information.

### BACKGROUND

Sensory substitution technology is technology for converting sensory information into other type sensory information. Recently, for visually-impaired persons who lose a visual function, sensory substitution technology for converting visual information into sensory information corresponding to another sense, which is not impaired, of a blind person has been developed.

Conventional sensory substitution technology for blind persons may replace an impaired visual organ of a blind person with a sensor such as a camera and performs sensory data processing of image information, obtained from the sensor, into other sensory modality information corresponding to a sense (an auditory sense or a tactile sense), which is not impaired, of the blind person. Therefore, blind persons may perceive a visual sense through sensory modality (an auditory sense or a tactile sense).

However, a number of conventional sensory substitution technologies for blind persons provide limited visual information such as a depth, a shape, and a size of an object displayed in an image to enable a blind person to perceive the visual information, but have a limitation in enabling a blind person to intuitively perceive color information displayed in an image.

### SUMMARY

Accordingly, the present invention provides a sensory substitution apparatus and method, which substitute color information about an image into a combination of various pieces of tactile information and provide a user with more intuitive sensory substitution in a process of substituting the color information about the image into the tactile information.

In one general aspect, a sensory substitution method includes: obtaining a color image by using a sensor device; converting color information about the color image, received from the sensor device, into tactile information by using a user terminal; and generating a tactile stimulus corresponding to the tactile information received from the user terminal by using a tactile output device.

In another general aspect, a sensory substitution apparatus includes: a sensor device configured to obtain a color image; a user terminal configured to convert color information about the color image, received from the sensor device, into tactile information; and a tactile output device configured to generate a tactile stimulus corresponding to the tactile information received from the user terminal.

In another general aspect, a sensory substitution method includes: obtaining an image displayed in a first color space by using a sensor device; converting first color information, included in the image received from the sensor device, into second color information displayed in a second color space and converting the second color information into tactile information by using a user terminal; and generating a tactile stimulus corresponding to the tactile information received from the user terminal by using a tactile output device.

Other features and aspects will be apparent from the following detailed description, the drawings, and the claims.

### BRIEF DESCRIPTION OF THE DRAWINGS

FIG. 1 is a block diagram of a sensory substitution apparatus according to an embodiment of the present invention.

FIG. 2 is a diagram illustrating a mapping relationship between a color sensation and a tactile sensation according to an embodiment of the present invention.

FIG. 3 is a diagram illustrating a mapping relationship between a hue value and thermal (temperature) intensity according to an embodiment of the present invention.

FIG. 4 is a diagram illustrating an implementation example of each device illustrated in FIG. 1.

FIG. 5 is a diagram illustrating a configuration of a tactile stimulus pad according to an embodiment of the present invention.

FIG. 6 is a flowchart illustrating a sensory substitution method according to an embodiment of the present invention.

### DETAILED DESCRIPTION OF EMBODIMENTS

Hereinafter, example embodiments of the present invention will be described in detail with reference to the accompanying drawings. Embodiments of the present invention are provided so that this disclosure will be thorough and complete, and will fully convey the concept of the present invention to one of ordinary skill in the art. Since the present invention may have diverse modified embodiments, preferred embodiments are illustrated in the drawings and are described in the detailed description of the present invention. However, this does not limit the present invention within specific embodiments and it should be understood that the present invention covers all the modifications, equivalents, and replacements within the idea and technical scope of the present invention. Like reference numerals refer to like elements throughout.

It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. In various embodiments of the disclosure, the meaning of ‘comprise’, ‘include’, or ‘have’ specifies a property, a region, a fixed number, a step, a process, an element and/or a component but does not exclude other properties, regions, fixed numbers, steps, processes, elements and/or components.

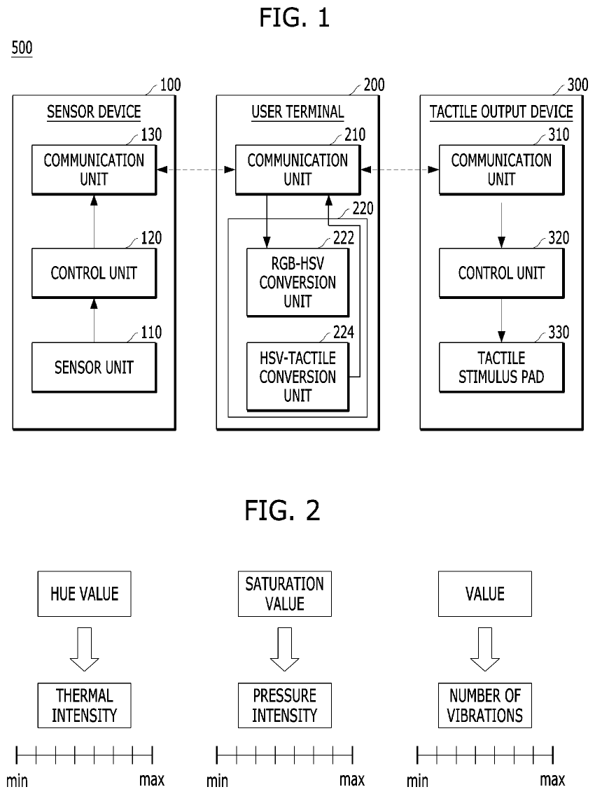

FIG. 1 is a block diagram of a sensory substitution apparatus 500 according to an embodiment of the present invention.

Referring to FIG. 1, the sensory substitution apparatus 500 according to an embodiment of the present invention may substitute color information about an object, included in an image captured by a sensor, into a combination of pieces of tactile stimulus information representing tactile stimulus factors such as heat, pressure, and a vibration and may transfer substituted information to a user, and thus, a visually-impaired user may intuitively perceive color information about an object included in an image on the basis of a tactile modality (heat, pressure, and a vibration).

To this end, the sensory substitution apparatus 500 according to an embodiment of the present invention may include a sensor device 100, a user terminal 200, and a tactile output device 300.

The sensor device 100 may be a device which replaces a visual function of a user, and the sensor device 100 may obtain and collect a color image and may transmit the collected image to the user terminal 200. To this end, the sensor device 100 may include a sensor unit 110, a control unit 120, and a communication unit 130. The sensor unit 110 may be an element which photographs an object to obtain a color image, and for example, may be a camera. The control unit 120 may control an operation of each of the sensor unit 110 and the communication unit 130 and may be implemented as at least one processor having a processing function. Also, the control unit 120 may process the color image, obtained by the sensor unit 110, into appropriate data which is to be transmitted to the user terminal 200. The communication unit 130 may support wireless communication, for transmitting a color image, obtained through processing by the control unit 120, to the user terminal 200. In order to support the wireless communication, the communication unit 130 may include a modem, an amplifier, a filter, and a frequency conversion component, which are appropriate. The wireless communication may be near-field wireless communication such as WiFi or Bluetooth.

The user terminal 200 may receive the color image from the sensor device 100 and may substitute (or convert) color information about a background or an object, included in the received color image, into tactile stimulus information. To this end, the user terminal 200 may include a communication unit 210 and a control unit 220. The communication unit 210 may support wireless communication, for receiving a color image from the sensor device 100. In order to support the wireless communication, the communication unit 210 may include a modem, an amplifier, a filter, and a frequency conversion component, which are appropriate. The wireless communication may be near-field wireless communication such as WiFi or Bluetooth. The control unit 220 may perform, by using the communication unit 210, data processing for substituting color information about an object, included in a color image received from the sensor device 100, into tactile stimulus information. The control unit 220 may include an RGB-HSV conversion unit 222 and an information (HSV-tactile) conversion unit 224 on the basis of a unit of data processing. Each of the RGB-HSV conversion unit 222 and the HSV-tactile conversion unit 224 may be a software module, a hardware module, or a combination thereof. In a case where each of the RGB-HSV conversion unit 222 and the HSV-tactile conversion unit 224 is implemented as a software module, the control unit 220 may include at least one processor for executing the software module. In a case where each of the RGB-HSV conversion unit 222 and the HSV-tactile conversion unit 224 is implemented as a software module, each of the RGB-HSV conversion unit 222 and the HSV-tactile conversion unit 224 may be a circuit logic equipped in a processor. The RGB-HSV conversion unit 222 may extract a hue value, a saturation value, and a value from color information (for example, red (R), green (G), and blue (B)) about a color image received from the sensor device 100. To this end, the RGB-HSV conversion unit 222 may convert an RGB value, expressed in an RGB color space, into an HSV value expressed in an HSV color space. A color space conversion algorithm (or a color space conversion function) for converting the RGB value into the HSV value may be used and the color space conversion algorithm (or the color space conversion function) may not be a feature of the present invention, and thus, its description may be the same as that of technology known to those skilled in the art.

The HSV-tactile conversion unit 224 may convert a hue value H, a saturation value S, and a value V, generated by the RGB-HSV conversion unit 222, into tactile information representing thermal intensity (or temperature intensity), tactile information representing pressure intensity (or vibration intensity), and tactile information representing the number of vibrations (or a vibration frequency). That is, the hue value H may be converted into the thermal intensity (or temperature intensity), the saturation value S may be converted into the pressure intensity (or a vibration amplitude, vibration intensity, or vibration strength), and the value V may be converted into the number of vibrations (or a vibration frequency).

Pieces of tactile information (thermal intensity, pressure intensity, and the number of vibrations) generated by the HSV-tactile conversion unit 224 may be transmitted to the tactile output device 300 through the communication unit 210.

The tactile output device 300 may receive the pieces of tactile information from the user terminal 200 and may combine the thermal intensity, the pressure intensity (or a vibration amplitude, vibration intensity, or vibration strength), and the number of vibrations corresponding to the received pieces of tactile information to transfer a complex tactile stimulus to the user.

To this end, the tactile output device 310 may include a communication unit 310, a control unit 320, and a tactile stimulus pad 330.

The communication unit 310 may support wireless communication, for receiving pieces of tactile information from the user terminal 200. In order to support the wireless communication, the communication unit 310 may include a modem, an amplifier, a filter, and a frequency conversion component, which are appropriate. The wireless communication may be near-field wireless communication such as WiFi or Bluetooth.

The control unit 320 may include at least one processor for controlling an overall operation of the tactile output device 300. Also, the control unit 320 may generate a control signal for controlling the tactile stimulus pad 330 so that the tactile stimulus pad 330 combines pieces of tactile information received from the user terminal 200 through the communication unit 310 to generate a complex tactile stimulus. Here, the control signal may be a pulse width modulation (PWM) signal.

The tactile stimulus pad 330 may generate the complex tactile stimulus in response to the control signal (a PWM signal) from the control unit 320.

Hereinafter, a method of converting color information into tactile information will be described in detail.

According to research on multi-sense perception mechanism of brain, it has been reported that brightness of visually perceived light, a size of an object, and a pitch and loudness of an auditorily perceived sound are psychophysically correlated therebetween and have a crossmodal correspondence relationship having a psychophysical correlation in a sensory perception relationship between a color, a temperature, and a vibration.

Correspondence between a visual stimulus and a tactile stimulus based on a color and color brightness corresponding to visual cues and heat and a vibration corresponding to tactile cues may be intuitively construed as the same visual information through certain training and learning even when visual stimulus information is converted into tactile stimulus information having a correspondence relationship therebetween. Therefore, based on crossmodal plasticity of brain, intuitiveness may be more reinforced in proportion to a learning time.

FIG. 2 is a diagram illustrating a mapping relationship between a color sensation and a tactile sensation according to an embodiment of the present invention.

Referring to FIG. 2, in a mapping relationship between color perception H, S, V and tactile perception according to an embodiment of the present invention, a hue value H may be fundamentally mapped to thermal (temperature) intensity, a saturation value S may be fundamentally mapped to pressure intensity, and a value V may be fundamentally mapped to the number of vibrations (or a vibration frequency).

In hue information, in a case where red, scarlet, and yellow providing a warm color sense are mapped to thermal (temperature) intensity, red may be mapped at a thermal (temperature) intensity level which enables the perception of hot feeling, scarlet may be mapped at a thermal (temperature) intensity level which enables the perception of warm feeling, and yellow may be mapped at a thermal (temperature) intensity level which enables the perception of lukewarm feeling.

In the hue information, in a case where green, blue, dark blue, and violet providing a cold color sense are mapped to thermal (temperature) intensity, green may be mapped at a thermal (temperature) intensity level which enables the perception of little cool feeling, blue may be mapped at a thermal (temperature) intensity level which enables the perception of cool feeling, dark blue may be mapped at a thermal (temperature) intensity level which enables the perception of cold feeling, and violet may be mapped at a thermal (temperature) intensity level which enables the perception of icy feeling.

A mapping relationship between the hue information and a thermal (temperature) intensity level may be described as an inversely proportional relationship. That is, as the hue value H increases, a thermal (temperature) intensity level may be lowered.

A temperature causing several cold and warm senses may be variable based on a stimulus environment (a skin state, a stimulus day time, and an individual difference) and a feature (a temperature, a variation speed, a period, a stimulus region, and a stimulus history) of a used stimulus, and thus, a mapping relationship between the hue value H and a thermal (temperature) intensity level may vary based on a person and an environment.

In a skin having no hair, a cold-sense temperature enabling pain to feel may be 10° C. to 15° C. and a warm-sense temperature may be 45° C. or more, and thus, the hue value H may be changed to a temperature level defined at a certain interval within a temperature range 15° C. to 45° C. of a temperature representing a cold sense and a warm sense.

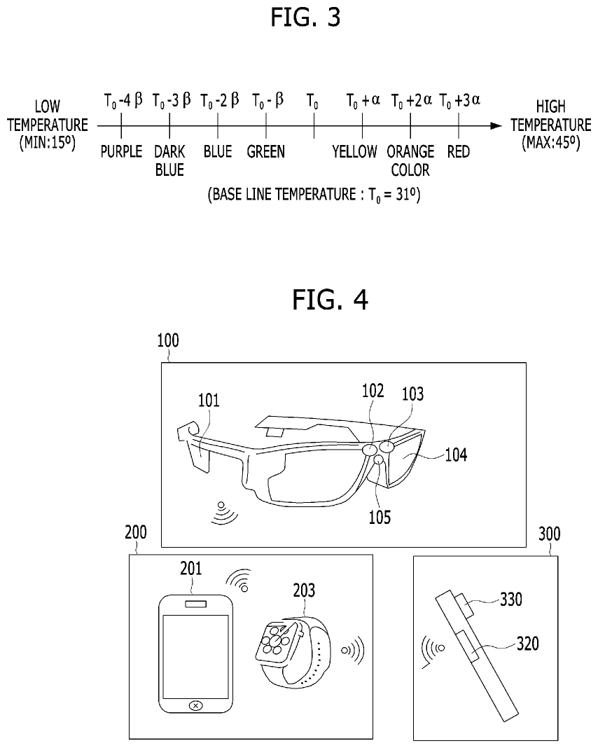

A temperature of 33° C. or more may be a temperature band which causes a warm sense to humans, and thus, a temperature 30° C. to 32° C. corresponding to an intermediate value allowing a cold sense and a warm sense not to feel may be set to a base line temperature. In such a temperature band, pressure intensity and the number of vibrations may be mapped to an achromatic color such as white or black. In the base line temperature, a high frequency vibration may be mapped to white, and a low frequency vibration may be mapped to black. In FIG. 3, an example of a mapping relationship between a hue value H and thermal (temperature) intensity is illustrated.

Saturation information representing purity of hue may be mapped to pressure intensity. When pressure intensity (vibration amplitude and vibration intensity) is high, the high pressure intensity may be mapped to a saturation value S where purity of hue is high, and low pressure intensity may be mapped to a saturation value S where purity of hue is low. That is, a saturation value S and a pressure intensity level may be described as having a proportional relationship therebetween.

Value information may be mapped to a vibration number (vibration frequency) level. Bright value (maximum value “0”) may be mapped to a vibration number level which enables the perception of a high frequency vibration, dark value (maximum value “1”) may be mapped to a vibration number level which enables the perception of a low frequency vibration, and brightness information about hue perceived by a cold sense and a warm sense may be perceived. That is, a mapping relationship between value V and a vibration number (vibration frequency) level may be described as having a proportional relationship therebetween.

A frequency band of a vibration may use a frequency band capable of being sensed by a finger of a human, and for example, may be divided and used based on a brightness level in a frequency band of 1 Hz to 350 Hz.

FIG. 4 is a diagram illustrating an implementation example of each device illustrated in FIG. 1.

Referring to FIG. 4, the sensor device 100 illustrated in FIG. 1 may be implemented as a glasses-shaped body. Also, a plurality of sensors may be equipped in the body. The plurality of sensors may include a wide angle camera 102 which captures a color image, a depth camera 103 which obtains depth information about an image, an infrared (IR) emitter 104 which includes a thin-film array for emitting infrared light, and an acceleration and angular speed sensor 1055 for measuring a direction of an eye-gaze. Additionally, a bone conduction speaker 101 for outputting audio information without hindering external sound information may be further equipped in the glasses-shaped body. Images obtained by the plurality of sensors may be transmitted to the user terminal 200 through a communication unit (300 of FIG. 1) which supports near-field wireless communication such as WiFi or Bluetooth.

The user terminal 200 may respectively convert a hue value H, a saturation value S, and a value V into pieces of tactile information including thermal intensity, pressure intensity, and the number of vibrations and may be implemented as a mobile device, having a processing function, such as a smartphone 201 or a smart watch 203 which transmits the tactile information to the tactile output device 300.

The tactile output device 300 may include a tactile stimulus pad 330 which combines the thermal intensity, the pressure intensity, and the number of vibrations received from the user terminal 200 on the basis of control by the control unit 320 to transfer a complex tactile stimulus to a user.

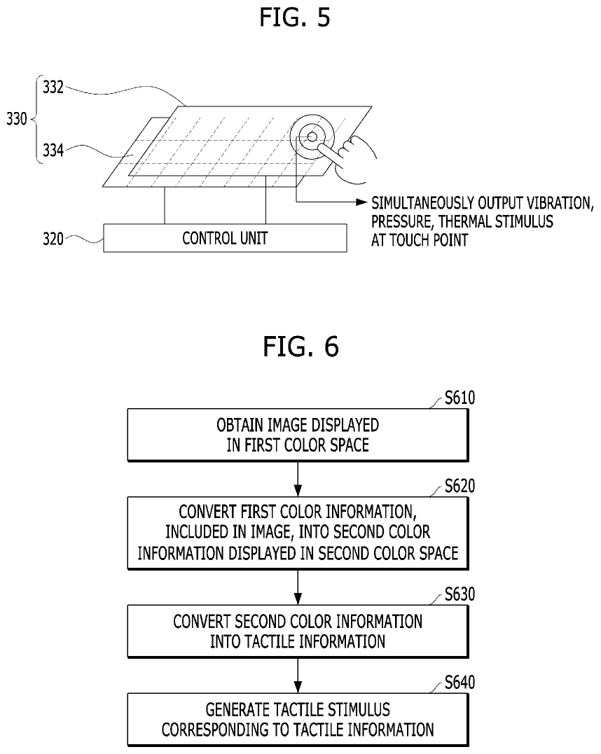

FIG. 5 is a diagram illustrating a configuration of a tactile stimulus pad 300 according to an embodiment of the present invention.

Referring to FIG. 5, the tactile stimulus pad 300 according to an embodiment of the present invention may include a physical force varying layer 332 and a heat varying layer 334, so as to simultaneously output a complex tactile stimulus including heat, pressure, and the number of vibrations at a finger touch point.

In order to generate the complex tactile stimulus, the physical force varying layer 332 may configure an upper layer, and the heat varying layer 334 may configure a lower layer.

The physical force varying layer 332 may generate a physical stimulus based on the number of vibrations and pressure (vibration intensity), and the heat varying layer 334 may generate a thermal stimulus (a temperature stimulus).

The heat varying layer 334 may be disposed under the physical force varying layer 332, and thus, a tactile stimulus where a tactile stimulus based on the number of vibrations, a tactile stimulus based on pressure, and a tactile stimulus based on heat (temperature) are combined may be generated at a point touched by a finger of a user.

The physical force varying layer 332 may include a tactile sensing function capable of measuring a position of a stimulus and may be implemented with an electrostatic force actuator, an electric active polymer actuator, a piezoelectric actuator, a linear resonance actuator, and a transparent thin-film actuator, for generating pressure and the number of vibrations of various types. The physical force varying layer 332 including various actuators may generate the number of vibrations and pressure (vibration intensity), which are variously adjusted based on pulse width modulation (PWM) control by the control unit 320.

The heat varying layer 334 may be a thermal conductive material layer which generates a distribution and a variation of a temperature on the basis of a heat generating or heat absorbing effect (Peltier effect). The heat varying layer 334 may include a polymer material or a metal material, which is high in thermal conductivity, and may be implemented as a thin film type.

The control unit 320 may perform heat generating control or heat absorbing control at a desired target temperature by controlling the supply of power. The control unit 320 may appropriately adjust the amount of supplied power and a power period, and thus, may perform heat generating control or heat absorbing control at various levels.

FIG. 6 is a flowchart illustrating a sensory substitution method according to an embodiment of the present invention.

Referring to FIG. 6, in step S610, the sensor device 100 may perform a process of obtaining an image displayed in a first color space. Here, the first color space may be an RGB color space. In this case, an image displayed in the RGB color space may be a color image including an RGB value. The obtained image may be transmitted to the user terminal 200 on the basis of a wireless communication scheme.

Subsequently, in step S620, the user terminal 200 or the RGB-HSV conversion unit 222 of the user terminal 200 may perform a process of converting first color information, included in the image received from the sensor device 100, into second color information displayed in a second color space. Here, the second color space may be CIELAB, an HSV (hue, saturation, and value) color space, or an HSI (hue, saturation, and intensity) color space. In the present embodiment, the second color space may be assumed to be an HSV color space, and thus, the second color information may include a hue (H) value, a saturation (S) value, and a value (V).

Subsequently, in step S630, the user terminal 200 or the HSV-tactile conversion unit 224 of the user terminal 200 may perform a process of converting the second color information H, S, and V into tactile information. In detail, the hue value H may be converted into a thermal (temperature) level, the saturation value S may be converted into a pressure level, and the value V may be converted into a vibration number level. A mapping table where a mapping relationship is previously defined may be used for respectively converting the hue value H, the saturation value S, and the value V into the thermal (temperature) level, the pressure level (or vibration intensity), and the vibration number level (a vibration frequency). According to the mapping table, the hue value and the thermal (temperature) level may be mapped to each other in an inversely proportional relationship, the saturation value and the pressure level may be mapped to each other in an inversely proportional relationship, and the value and the vibration number level may be mapped to each other in an inversely proportional relationship. The user terminal 200 may transmit tactile information, converted (or extracted) from the second color information H, S, and V, to the tactile output device 300 on the basis of a wireless communication scheme.

Subsequently, in step S640, the tactile output device 300 may generate a tactile stimulus corresponding to the tactile information received from the user terminal 200. A method of generating the tactile stimulus, as described above with reference to FIG. 5, may generate a thermal stimulus corresponding to the thermal (temperature) level by using the heat varying layer 334 including a thermal conductive material and may generate a pressure stimulus corresponding to the pressure level (vibration intensity) and a vibration number stimulus corresponding to the vibration number level by using the physical force varying layer 332 including an actuator.

As described above, in the embodiments of the present invention, color information about an image may be mapped to and converted into complex tactile information including a combination of physical tactile stimulus factors of heat, vibration, and pressure, and thus, a user may intuitively perceive color information about a background or an object in the image by using a tactile modality. Accordingly, in the embodiments of the present invention, an impaired visual function of a blind person may be enhanced, and thus, continuous economic activity and life quality may be enhanced.

The sensory substitution apparatus according to the embodiments of the present invention may substitute color information about an image into tactile information including a combination of physical tactile stimulus factors including heat, pressure, and a vibration and may transfer the tactile information to a user, and thus, the user may efficiently and intuitively perceive the color information about the image on the basis of the combination of the physical tactile stimulus factors.

A number of exemplary embodiments have been described above. Nevertheless, it will be understood that various modifications may be made. For example, suitable results may be achieved if the described techniques are performed in a different order and/or if components in a described system, architecture, device, or circuit are combined in a different manner and/or replaced or supplemented by other components or their equivalents. Accordingly, other implementations are within the scope of the following claims.

What is claimed is:

1. A sensory substitution method comprising:
obtaining a color image by using a sensor device;
converting color information about the color image, received from the sensor device, into tactile information by using a user terminal; and
generating a tactile stimulus corresponding to the tactile information received from the user terminal by using a tactile output device.

2. The sensory substitution method of claim 1, wherein the converting comprises:
extracting a hue value, a saturation value, and a value, displayed in an HSV color space, from the color information; and
converting the hue value, the saturation value, and the value into the tactile information including a thermal (temperature) level, a pressure level, and a vibration number level.

3. The sensory substitution method of claim 2, wherein the hue value and the thermal (temperature) level are mapped to each other in an inversely proportional relationship.

4. The sensory substitution method of claim 2, wherein the saturation value and the pressure level are mapped to each other in an inversely proportional relationship.

5. The sensory substitution method of claim 2, wherein the value and the vibration number level are mapped to each other in an inversely proportional relationship.

6. The sensory substitution method of claim 2, wherein the pressure level is vibration intensity.

7. The sensory substitution method of claim 1, wherein the generating comprises generating the tactile stimulus including a thermal (temperature) stimulus, a pressure stimulus, and a vibration number stimulus.

8. The sensory substitution method of claim 1, wherein the generating comprises:
generating a thermal stimulus by using a thermal conductive material;
generating a pressure stimulus corresponding to vibration intensity by using a physical force varying layer including an actuator; and
generating a vibration number stimulus by using the physical force varying layer.

9. The sensory substitution method of claim 1, wherein the generating comprises generating a complex tactile stimulus where the thermal stimulus, the pressure stimulus, and the vibration number stimulus are combined, based on a stack structure where the heat varying layer and the physical force varying layer are stacked.

10. A sensory substitution apparatus comprising:
a sensor device configured to obtain a color image;
a user terminal configured to convert color information about the color image, received from the sensor device, into tactile information; and
a tactile output device configured to generate a tactile stimulus corresponding to the tactile information received from the user terminal.

11. The sensory substitution apparatus of claim 10, wherein the sensor device comprises:
a camera configured to capture the color image; and
a communication unit configured to transmit the color image to the user terminal.

12. The sensory substitution apparatus of claim 10, wherein the user terminal comprises:
a communication unit configured to receive the color image;
an RGB-HSV conversion unit configured to convert an RGB value, included in the received color image, into a hue value, a saturation value, and a value; and
an HSV-tactile conversion unit configured to convert the hue value, the saturation value, and the value into the tactile information including a thermal (temperature) level, a pressure level, and a vibration number level by using a mapping table.

13. The sensory substitution apparatus of claim 12, wherein
the HSV-tactile conversion unit converts the hue value into the thermal (temperature) level, and
the hue value and the thermal (temperature) level are inversely proportional.

14. The sensory substitution apparatus of claim 12, wherein
the HSV-tactile conversion unit converts the saturation value into the pressure level, and
the saturation value and the pressure level are inversely proportional.

15. The sensory substitution apparatus of claim 12, wherein
the HSV-tactile conversion unit converts the value into the vibration number level, and
the value and the vibration number level are inversely proportional.

16. The sensory substitution apparatus of claim 10, wherein the tactile output device comprises:
a communication unit configured to receive the tactile information from the user terminal;
a control unit configured to generate a control signal for generating a tactile stimulus corresponding to the tactile information; and
a tactile stimulus pad configured to generate the tactile stimulus on the basis of the control signal.

17. The sensory substitution apparatus of claim 16, wherein the tactile stimulus pad comprises:
a heat varying layer configured to generate a temperature corresponding to a thermal (temperature) level included in the tactile information on the basis of the control signal; and
a physical force varying layer stacked on the heat varying layer and configured to vibrate based on number of vibrations corresponding to a vibration number level and vibration intensity corresponding to a pressure level, which are included in the tactile information.

18. A sensory substitution method comprising:
obtaining an image displayed in a first color space by using a sensor device;
converting first color information, included in the image received from the sensor device, into second color information displayed in a second color space and converting the second color information into tactile information by using a user terminal; and
generating a tactile stimulus corresponding to the tactile information received from the user terminal by using a tactile output device.

19. The sensory substitution method of claim 18, wherein the converting of the second color information into tactile information comprises:
converting the first color information, including an RGB value displayed in an RGB color space, into an HSV value displayed in an HSV color space; and
converting the HSV value into the tactile information including a temperature level, a pressure level corresponding to vibration intensity, and a vibration number level corresponding to a vibration frequency.
